(19) United States
(12) Patent Application Publication
(10) Pub. No.: US 2011/0144167 A1
(43) Pub. Date: Jun. 16, 2011

(54) Prolyl Hydroxylase Inhibitors

(75) Inventor: Rosanna Tedesco, Collegeville, PA (US)

(73) Assignee: SMITHKLINE BEECHAM CORPORATION, Philadelphia, PA (US)

(21) Appl. No.: 13/059,493

(22) PCT Filed: Aug. 21, 2009
(86) PCT No.: PCT/US09/54576

§ 371 Date: Feb. 17, 2011

Related U.S. Application Data
(60) Provisional application No. 61/091,443, filed on Aug. 25, 2008.

(51) Int. Cl. A61K 31/4412; C07D 213/69; A61P 7/06

(52) U.S. Cl. 514/348; 546/296

(57) ABSTRACT

The invention described herein relates to certain pyrimidinedione N-substituted glycine derivatives of formula (I)

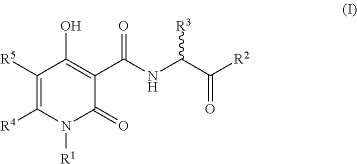

which are antagonists of HIF prolyl hydroxylases and are useful for treating diseases benefiting from the inhibition of this enzyme, anemia being one example.

### FIELD OF THE INVENTION

This invention relates to certain heteroaromatic N-substituted glycine derivatives that are inhibitors of HIF prolyl hydroxylases, and thus have use in treating diseases benefiting from the inhibition of this enzyme, anemia being one example.

### BACKGROUND OF THE INVENTION

Anemia occurs when there is a decrease or abnormality in red blood cells, which leads to reduced oxygen levels in the blood. Anemia occurs often in cancer patients, particularly those receiving chemotherapy. Anemia is often seen in the elderly population, patients with renal disease, and in a wide variety of conditions associated with chronic disease.

Frequently, the cause of anemia is reduced erythropoietin (Epo) production resulting in prevention of erythropoiesis (maturation of red blood cells). Epo production can be increased by inhibition of prolyl hydroxylases that regulate hypoxia inducible factor (HIF).

One strategy to increase erythropoietin (Epo) production is to stabilize and thus increase the transcriptional activity of the HIF. HIF-alpha subunits (HIF-1alpha, HIF-2alpha, and HIF-3alpha) are rapidly degraded by proteosome under normoxic conditions upon hydroxylation of proline residues by prolyl hydroxylases (EGLN1, 2, 3). Proline hydroxylation allows interaction with the von Hippel Lindau (VHL) protein, a component of an E3 ubiquitin ligase. This leads to ubiquitination of HIF-alpha and subsequent degradation. Under hypoxic conditions, the inhibitory activity of the prolyl hydroxylases is suppressed, HIF-alpha subunits are therefore stabilized, and HIF-responsive genes, including Epo, are transcribed. Thus, inhibition of prolyl hydroxylases results in increased levels of HIF-alpha and thus increased Epo production.

The compounds of this invention provide a means for inhibiting these hydroxylases, increasing Epo production, and thereby treating anemia. Ischemia, stroke, and cytoprotection may also benefit by administering these compounds.

### SUMMARY OF THE INVENTION

In the first instance, this invention relates to a compound of formula (I):

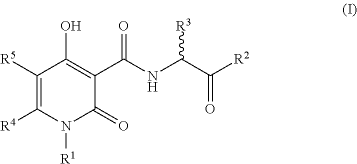

wherein:

R¹ is H, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;

R² is —NR⁶R⁷ or —OR⁸;

R³ is H or C₁-C₄alkyl;

R⁴ and R⁵ are each independently selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;

R⁶ and R⁷ are each independently selected from the group consisting of hydrogen, C₁-C₁₀ alkyl, C₂-C₁₀ alkenyl, C₂-C₁₀ alkynyl, C₃-C₈ cycloalkyl, C₃-C₈ heterocycloalkyl, aryl and heteroaryl;

R⁸ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;

where any carbon or heteroatom of R¹, R³, R⁴, R⁵, R⁶, R⁷, R⁸ is unsubstituted or, where possible, is substituted with one or more substituents independently selected from C₁-C₆ alkyl, aryl, heteroaryl, halogen, —OR⁹, —NR⁶R⁷, cyano, nitro, —C(O)R⁹, —C(O)OR⁹, —SR⁹, —S(O)R⁹, —S(O)₂R⁹, —NR⁶R⁷, —CONR⁶R⁷, —N(R⁶)C(O)R⁹, —N(R⁶)C(O)OR⁹, —OC(O)NR⁶R⁷, —N(R⁶)C(O)NR⁶R⁷, —SO₂NR⁶R⁷, —N(R⁶)SO₂R⁹, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R⁹ is hydrogen, C₁-C₁₀alkyl, C₂-C₁₀alkenyl, C₂-C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;

or a pharmaceutically acceptable salt or solvate thereof.

In a second aspect of the present invention, there is provided a compound of formula (I) or a salt or solvate thereof for use in mammalian therapy, e.g. treating amenia. An example of this therapeutic approach is that of a method for treating anemia caused by increasing the production of erythropoietin (Epo) by inhibiting HIF prolyl hydroxylases comprising administering a compound of formula (I) to a patient in need thereof, neat or admixed with a pharmaceutically acceptable excipient, in an amount sufficient to increase production of Epo.

In a third aspect of the present invention, there is provided a pharmaceutical composition comprising a compound of formula (I) or a salt, solvate, or the like thereof, and one or more of pharmaceutically acceptable carriers, diluents and excipients.

In a fourth aspect, there is provided the use of a compound of formula (I) or a salt or solvate thereof in the preparation of a medicament for use in the treatment of a disorder mediated by inhibiting HIF prolyl hydroxylases, such as an anemia, that can be treated by inhibiting HIF prolyl hydroxylases.

### DETAILED DESCRIPTION OF THE INVENTION

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁-C₄ alkyl” and “C₁-C₁₀ alkyl” refers to an alkyl group having at least 1 and up to 4 or 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “C₃-C₈cycloalkyl” refers to a non-aromatic cyclic hydrocarbon ring having from three to eight carbon atoms. Exemplary “C₃-C₈ cycloalkyl” groups useful in the present invention include, but are not limited to, cyclopropyl, cyclobutyl, cyclopentyl, cyclohexyl, cycloheptyl and cyclooctyl.

The term “C₅-C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈ heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,3-dioxane, piperidine, piperazine, 2,4-piperazinedione, pyrrolidine, imidazolidine, pyrazolidine, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl and the like.

“Heteroaryl” means an optionally substituted aromatic monocyclic ring or polycarbocyclic fused ring system wherein at least one ring complies with Hückel's Rule, has the specified number of ring atoms, and that ring contains at least one heteratom selected from N, O, and/or S. Examples of “heteroaryl” groups include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl.

The term “optionally” means that the subsequently described event(s) may or may not occur, and includes both event(s), which occur, and events that do not occur.

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically-acceptable acid addition salts by treatment with a suitable acid. Suitable acids include pharmaceutically-acceptable inorganic acids amd pharmaceutically-acceptable organic acids. Representative pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Compounds of particular interest include those wherein:

R¹ is hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;

R² is —OR¹¹;

R³ is H or C₁-C₄alkyl;

R⁴ and R⁵ are each independently selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;

R⁶ and R⁷ are each independently selected from the group consisting of hydrogen, C₁-C₁₀ alkyl, C₂-C₁₀ alkenyl, C₂-C₁₀ alkynyl, C₃-C₈ cycloalkyl, C₃-C₈ heterocycloalkyl, aryl and heteroaryl;

R⁸ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;

where any carbon or heteroatom of R¹, R², R³, R⁴, R⁵ is unsubstituted or, where possible, is substituted with one or more substituents independently selected from C₁-C₆ alkyl, aryl, heteroaryl, halogen, —OR⁹, —NR⁶R⁷, cyano, nitro, —C(O)R⁹, —C(O)OR⁹, —SR⁹, —S(O)R⁹, —S(O)₂R⁹, —NR⁶R⁷, —CONR⁶R⁷, —N(R⁶)C(O)R⁹, —N(R⁶)C(O)OR⁹, —OC(O)NR⁶R⁷, —N(R⁶)C(O)NR⁶R⁷, —SO₂NR⁶R⁷, —N(R⁶)SO₂R⁹, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R⁹ is hydrogen, C₁-C₁₀alkyl, C₂-C₁₀alkenyl, C₂-C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl.

Of further interest are those compounds where:

R¹ is selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;

R² is —OR⁸;

R³ is H;

R⁴ and R⁵ are each independently selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;

R⁶ and R⁷ are each independently selected from the group consisting of hydrogen, C₁-C₁₀ alkyl, C₂-C₁₀ alkenyl, C₂-C₁₀ alkynyl, C₃-C₈ cycloalkyl, C₃-C₈ heterocycloalkyl, aryl and heteroaryl;

R⁸ is H or a cation;

where any carbon or heteroatom of R¹, R², R³, R⁴ and R⁵ is unsubstituted or, where possible, is substituted with one or more substituents independently selected from C₁-C₆ alkyl, aryl, heteroaryl, halogen, —OR⁹, —NR⁶R⁷, cyano, nitro, —C(O)R⁹, —C(O)OR⁹, —SR⁹, —S(O)R⁹, —S(O)₂R⁹, —NR⁶R⁷, —CONR⁶R⁷, —N(R⁶)C(O)R⁹, —N(R⁶)C(O)OR⁹, —OC(O)NR⁶R⁷, —N(R⁶)C(O)NR⁶R⁷, —SO₂NR⁶R⁷, —N(R⁶)SO₂R⁹, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R¹² is hydrogen, C₁-C₁₀alkyl, C₂-C₁₀alkenyl, C₂-C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;

or a pharmaceutically acceptable salt thereof.

Processes for preparing the compound of formula (I) are also within the ambit of this invention. To illustrate, a process for preparing a compound of formula (I)

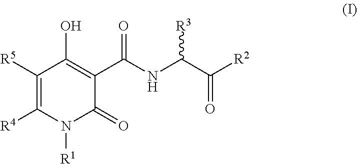

wherein R¹, R², R³, R⁴ and R⁵ are the same as defined above for formula (I), the process comprising treating a compound of formula A:

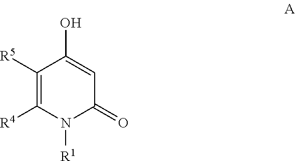

wherein R¹, R⁴ and R⁵ are the same as for those groups in formula (I) with an ethyl 2-isocyanatocarboxylate and an appropriate base, such as di-isopropylethylamine, in an appropriate solvent, such as dichloromethane, under either conventional thermal conditions or by microwave irradiation, to form a compound of formula (I) where R² is —OEt; or a process for preparing a compound of formula (I) wherein R¹, R², R³, R⁴ and R⁵ are the same as defined above for formula (I), the process comprising treating a compound of formula B:

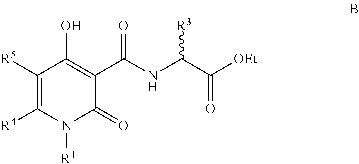

wherein R¹, R³, R⁴ and R⁵ are the same as for those groups in formula (I) with an alkali such as sodium hydroxide, in an appropriate solvent, such as aqueous ethanol, at a suitable temperature such as room temperature, to form a compound of formula (I) where R² is —OH;

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain of the compounds described herein may contain one or more chiral atoms, or may otherwise be capable of existing as two enantiomers. The compounds claimed below include mixtures of enantiomers as well as purified enantiomers or enantiomerically enriched mixtures. Also included within the scope of the invention are the individual isomers of the compounds represented by formula (I), or claimed below, as well as any wholly or partially equilibrated mixtures thereof. The present invention also covers the individual isomers of the claimed compounds as mixtures with isomers thereof in which one or more chiral centers are inverted. Also, it is understood that any tautomers and mixtures of tautomers of the claimed compounds are included within the scope of the compounds of formula (I) as disclosed herein above or claimed herein below.

Where there are different isomeric forms they may be separated or resolved one from the other by conventional methods, or any given isomer may be obtained by conventional synthetic methods or by stereospecific or asymmetric syntheses.

While it is possible that, for use in therapy, a compound of formula (I), as well as salts, solvates and the like, may be administered as a neat preparation, i.e. no additional carrier, the more usual practice is to present the active ingredient confected with a carrier or diluent. Accordingly, the invention further provides pharmaceutical compositions, which includes a compound of formula (I) and salts, solvates and the like, and one or more pharmaceutically acceptable carriers, diluents, or excipients. The compounds of formula (I) and salts, solvates, etc, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates etc, with one or more pharmaceutically acceptable carriers, diluents or excipients.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavouring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of formula (I) for the treatment of anemia will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, etc., may be determined as a proportion of the effective amount of the compound of formula (I) per se. It is envisaged that similar dosages would be appropriate for treatment of the other conditions referred to above.

### DEFINITIONS

rt—room temperature
DMF—dimethylformamide
DMA—dimethylacetamide
THF—tetrahydrofuran
DBU—1,8-diazabicyclo[5.4.0]undec-7-ene
TFA—Trifluoroacetic acid
PyBOP—Benzotriazole-1-yl-oxy-tris-pyrrolidino-phosphonium hexafluorophosphate

### Chemical Background:

The compounds of this invention may be made by a variety of methods, including standard chemistry. Any previously defined variable will continue to have the previously defined meaning unless otherwise indicated. Illustrative general synthetic methods are set out below and then specific compounds of the invention as prepared are given in the examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognize if a stereocenter exists in compounds of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

### Illustrated Methods of Preparation

Treatment of an appropriately substituted ketoester and urethane with POCl₃ gave the substituted oxazine, which was substituted at the nitrogen for example by treatment with NaH and an appropriate bromide. Reaction with diethylmalonate in the presence of NaH, followed by reaction with sodium glycinate provided the compounds of the invention.

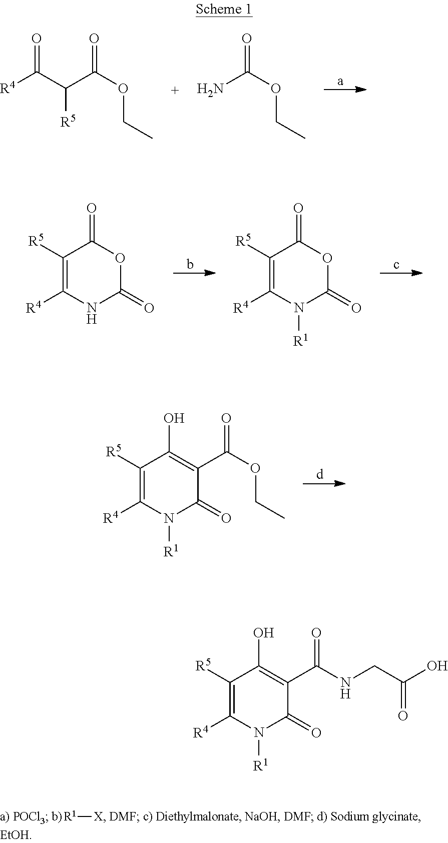

### Experimentals

### Example 1

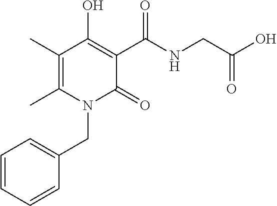

### N-{[4-hydroxy-5,6-dimethyl-2-oxo-1-(phenylmethyl)-1,2-dihydro-3-pyridinyl]carbonyl}glycine

1a) 4,5-dimethyl-2H-1,3-oxazine-2,6(3H)-dione. A mixture of urethane (9 g, 101 mmol) and ethyl 2-methylacetoacetate (14.3 ml, 101 mmol) in phosphorus oxychloride (23.8 ml, 253 mmol) was stirred at 95° C. for 2 h. The reaction mixture was cooled to rt and poured over ice/water and left overnight. It was then extracted with EtOAc (2×50 mL). The organics were washed with brine, dried over Na₂SO₄ and the solvent evaporated off. The residue was treated with Et₂O forming a solid which was collected and triturated from hexanes/EtOAc 1:1 to give the dione as a red powder (2.03 g, 14.4 mmol, 14% yield).

¹H-NMR (400 MHz, DMSO-d₆) δ ppm 11.31 (br. s., 1H), 2.06 (s, 3H), 1.77 (s, 3H)

1b) 4-Hydroxy-5,6-dimethyl-2-oxo-1-(phenylmethyl)-1,2-dihydro-3-pyridinecarboxylic acid. To a suspension of sodium hydride (128 mg, 3.19 mmol) N,N-dimethylacetamide (DMA) (2 ml) was added 4,5-dimethyl-2H-1,3-oxazine-2,6(3H)-dione (300 mg, 2.13 mmol) in portions. The reaction mixture was stirred at room temperature for 5 minutes, then treated with benzyl bromide (0.379 mL, 3.19 mmol) and stirred at 85-87° C. for 2 h. After cooling it was poured onto ice. The aqueous solution was extracted with ethyl acetate (2×100 ml), the organic layers combined and washed with brine (100 ml), dried over sodium sulfate, filtered and solvent evaporated off. The residue was azeotroped with toluene (3×), dissolved in THF and added to a mixture of NaH (341 mg, 8.52 mmol) and diethylmalonate (0.647 mL, 4.26 mmol) in THF (3 mL). The mixture was stirred at reflux for 2.5 h, cooled, poured into diluted HCl and stirred overnight. It was then extracted with EtOAc, the organic layer dried over Na₂SO₄ and the solvent removed by evaporation. The residue was purified by RP-HPLC to afford the title acid as a yellow glass (70 mg, 0.26 mmol, 12% yield).

¹H-NMR (400 MHz, CHLOROFORM-d) δ ppm 15.71 (br. s., 1H), 13.96 (br. s., 1H), 6.92-7.56 (m, 5H), 5.42 (br. s., 2H), 2.37 (s, 3H), 2.11 (s, 3H). LCMS (ES⁺) m/z 274 (MH⁺).

1c) N-{[4-hydroxy-5,6-dimethyl-2-oxo-1-(phenylmethyl)-1,2-dihydro-3-pyridinyl]carbonyl}glycine. A mixture of 4-hydroxy-5,6-dimethyl-2-oxo-1-(phenylmethyl)-1,2-dihydro-3-pyridinecarboxylic acid (70 mg, 0.26 mmol), glycine ethyl ester hydrochloride (39 mg, 0.28 mmol), PyBOP (135 mg, 0.26 mmol), triethyl amine (0.043 mL, 0.31 mmol) and diisopropylethylamine (0.054 mL, 0.31 mmol) in CH₂Cl₂ (7 mL) was stirred at rt for 20 h. It was then diluted with CH₂Cl₂ and washed with water, 1N HCl, water, brine, dried over Na₂SO₄ and the solvent was evaporated. The residue was dissolved in THF (2 mL) and 1M NaOH (2 mL) and refluxed for 1.5 h. The solvent was partially evaporated at reduced pressure, water added and the solution extracted with EtOAc. The aqueous solution was acidified by the addition of 1N HCl. A precipitated formed and was collected by filtration, washed with water and dried to afford the title compound (26 mg, 0.079 mmol, 30% yield) as a white powder.

¹H-NMR (400 MHz, DMSO-d₆) δ ppm 16.16 (s, 1H), 12.85 (br. s., 1H), 10.54 (t, J=5.56 Hz, 1H), 7.34 (t, J=7.33 Hz, 2H), 7.22-7.29 (m, 1H), 7.12 (d, J=7.07 Hz, 2H), 5.37 (br. s., 2 H), 4.07 (d, J=5.56 Hz, 2H), 2.29 (s, 3H), 1.97 (s, 3H). LCMS (ES⁺) m/z 331 (MH⁺).

### Example 2

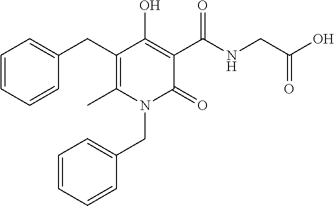

### N-{[4-hydroxy-6-methyl-2-oxo-1,5-bis(phenylmethyl)-1,2-dihydro-3-pyridinyl]carbonyl}glycine

2a) 4-Methyl-5-(phenylmethyl)-2H-1,3-oxazine-2,6(3H)-dione. A mixture of urethane (5 g, 56 mmol) and ethyl 2-(phenylmethyl)acetoacetate (12 ml, 56 mmol) in phosphorus oxychloride (11 ml, 118 mmol) was stirred at 95° C. for 2 h. The reaction mixture was cooled to rt and poured over ice/water and left to rest overnight. It was then extracted with EtOAc (2×50 mL). The organics were washed with brine, dried over Na₂SO₄ and solvent evaporated off and the residue filtered through silica gel (CH₂Cl₂ as eluent). Solvent was removed and the residue was treated with hot Et₂O, cooled, collected by filtration, washed with Et₂O and dried to give the title dione as an off white powder (4.3 g, 20 mmol, 35% yield).

¹H-NMR (400 MHz, DMSO-d₆) δ ppm 11.45 (s, 1H), 7.06-7.36 (m, 5H), 3.61 (s, 2H), 1.98-2.20 (m, 3H).

2b) 4-Methyl-3,5-bis(phenylmethyl)-2H-1,3-oxazine-2,6(3H)-dione. 4-Methyl-5-(phenylmethyl)-2H-1,3-oxazine-2,6(311)-dione (1.0 mg, 4.6 mmol) was added to a suspension of sodium hydride (203 mg, 5.1 mmol) in N,N-dimethylacetamide (8 ml) cooled to 0° C. The reaction mixture was stirred at room temperature for 20 minutes and then treated with benzyl bromide (0.574 ml, 5.1 mmol). The mixture was stirred at rt for 18 h, then poured onto ice. The precipitate was collected, washed with water, then hexanes. The solid collected was dried to give the title dione as a yellow powder (990 mg, 3.22 mmol, 70% yield).

¹H-NMR (400 MHz, DMSO-d₆) δ ppm 7.12-7.47 (m, 10H), 5.11 (s, 2H), 3.72 (s, 2H), 2.15 (s, 3H)

2c) N-{[4-hydroxy-6-methyl-2-oxo-1,5-bis(phenylmethyl)-1,2-dihydro-3-pyridinyl]carbonyl}glycine. DBU (0.48 ml, 3.3 mmol) was added to diethylmalonate (0.24 ml, 1.6 mmol) and 4-methyl-3,5-bis(phenylmethyl)-2H-1,3-oxazine-2,6(3H)-dione (450 mg, 1.5 mmol) and the mixture was then irradiated in a microwave at 150° C. for 20 min. Glycine (164 mg, 2.19 mmol) was then added and the mixture was irradiated at 155° C. for 25 min in the microwave. It was poured onto 1N HCl and extracted with EtOAc. The organic layer was washed with brine, dried over Na₂SO₄ and the solvent evaporated. The residue was purified by RP-HPLC (30 to 95% AcCN in H₂O plus 0.1% TFA) to give the title glycine as a yellow powder (44 mg, 0.108 mmol, 7% yield).

¹H NMR (400 MHz, DMSO-d₆) δ ppm 16.33 (s, 1H), 12.89 (br. s., 1H), 10.52 (t, J=5.68 Hz, 1 H), 7.35 (t, J=7.45 Hz, 2H), 7.26 (t, J=7.33 Hz, 3H), 7.08-7.19 (m, 5H), 5.38 (br. s., 2H), 4.08 (d, J=5.56 Hz, 2H), 3.89 (s, 2H), 2.26 (s, 3H). LCMS (ES⁺) m/z 407 (MH⁺).

### Example 3

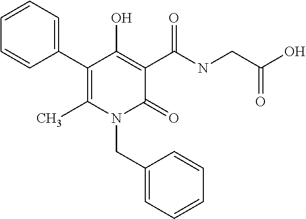

### N-{[4-hydroxy-6-methyl-2-oxo-5-phenyl-1-(phenylmethyl)-1,2-dihydro-3-pyridinyl]carbonyl}glycine

3a) 4-methyl-5-(phenyl)-2H-1,3-oxazine-2,6(3H)-dione. A mixture of urethane (2.2 g, 24.2 mmol) and ethyl 2-phenylacetoacetate (5.0 g, 24.2 mmol) in phosphorus oxychloride (5 ml, 53.6 mmol) was stirred at 95° C. for 1 h and 45 min. The reaction mixture was cooled to rt and poured over ice/water and left to rest overnight. A solid formed and was collected, washed with water, air dried and washed with Et₂O to give the title dione (3.74 g, 18.4 mmol, 76% yield). The washed solid was used as is in the next step.

¹H-NMR (400 MHz, DMSO-d₆) δ ppm 11.66 (s, 1H), 7.31-7.46 (m, 3H), 7.21-7.31 (m, 2H), 1.98 (s, 3H)

3b) 4-Methyl-5-phenyl-3-(phenylmethyl)-2H-1,3-oxazine-2,6(3H)-dione. 4-Methyl-5-phenyl-2H-1,3-oxazine-2,6(3H)-dione (1.0 mg, 4.9 mmol) was added portionwise to a suspension of sodium hydride (216 mg, 5.4 mmol) in N,N-dimethylacetamide (8 ml) cooled to 0° C. The reaction mixture was stirred at room temperature for 20 minutes and then treated with benzyl bromide (0.643 ml, 5.4 mmol). The mixture was stirred at rt for 18 h, then poured onto ice/water. The precipitate was collected by filtration, washed with water and then hexanes, and dried to provide the title dione as an off white powder (1.24 g, 4.22 mmol, 86% yield). This materials was used in the next step without further purification.

¹H NMR (400 MHz, DMSO-d₆) δ ppm 7.22-7.51 (m, 10H), 5.15 (s, 2H), 2.03 (s, 3H).

3c) Ethyl 4-hydroxy-6-methyl-2-oxo-5-phenyl-1-(phenylmethyl)-1,2-dihydro-3-pyridinecarboxylate. Sodium hydride (164 mg, 4.09 mmol) was suspended in DMA and the mixture was cooled to 0° C. Diethylmalonate (0.621 ml, 4.09 mmol) was added dropwise and stirring was continued for 10 minutes, then 4-methyl-5-phenyl-3-(phenylmethyl)-2H-1,3-oxazine-2,6(3H)-dione (1.0 mg, 3.41 mmol) was added. The reaction mixture was stirred at 120° C. for 3.5 hours, then cooled to 70° C. and treated with 3.0 mL AcOH. After stirring for 10 minutes it was cooled to rt, and the precipitate was collected and washed with water and hexanes. The residue was dissolved in CH₂Cl₂ and washed with an aq. sat. sol. of NaHCO₃, water and brine, then dried over Na₂SO₄ and the solvent was evaporated. The residue was dried under vacuum and triturated from Et₂O to give the title ester as a pale pink powder (685 mg, 1.88 mmol, 55% yield).

¹H NMR (400 MHz, DMSO-d₆) δ ppm 13.29 (s, 1H), 7.32-7.46 (m, 5H), 7.21-7.30 (m, 3H), 7.16 (d, J=7.33 Hz, 2H), 5.32 (br. s., 2H), 4.32 (q, J=7.07 Hz, 2H), 2.06 (s, 3H), 1.29 (t, 3 H). LCMS (ES⁺) m/z 364 (MH⁺).

3d) N-{[4-hydroxy-6-methyl-2-oxo-5-phenyl-1-(phenylmethyl)-1,2-dihydro-3-pyridinyl]carbonyl}glycine. Sodium glycinate (320 mg, 3.3 mmol) was added to a solution of ethyl 4-hydroxy-6-methyl-2-oxo-5-phenyl-1-(phenylmethyl)-1,2-dihydro-3-pyridinecarboxylate (680 mg, 1.65 mmol) in methoxyethanol (7 ml). The mixture was stirred at reflux for 1.5 h, cooled, then poured onto H₂O and extracted with EtOAc. The aqueous layer was acidified by the addition of 6 N HCl (pH=2). The solid was collected, washed with H₂O and dried. More solid was collected from the filtrate to afford a total of 525 mg of title glycine containing about 3% impurities by H-NMR. Then 240 mg of product were recrystallized from EtOAc/hexanes 2.5:1 to afford the title compound (154 mg) as white crystals.

¹H NMR (400 MHz, DMSO-d₆) δ ppm 16.03 (s, 1H), 12.85 (s, 1H), 10.49 (t, J=5.56 Hz, 1H), 7.32-7.46 (m, 5H), 7.21-7.31 (m, 3H), 7.18 (d, J=7.33 Hz, 2H), 5.40 (br. s., 2H), 4.07 (d, J=5.81 Hz, 2H), 2.11 (s, 3H). LCMS (ES⁺) m/z 393 (MH⁺).

### Example 4

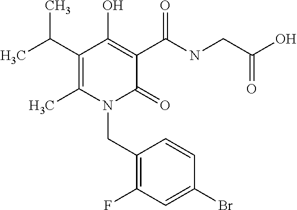

### N-{[1-[(4-bromo-2-fluorophenyl)methyl]-4-hydroxy-6-methyl-5-(1-methylethyl)-2-oxo-1,2-dihydro-3-pyridinyl]carbonyl}glycine

4a) 4-methyl-5-(1-methylethyl)-2H-1,3-oxazine-2,6(3H)-dione. A mixture of urethane (2 g, 22.45 mmol) and ethyl isopropylacetoacetate (4.02 ml, 22.45 mmol) in phosphorus oxychloride (5 ml, 53.6 mmol) was stirred at 95° C. for 2.5 h. The reaction mixture was cooled to rt and poured over ice/water and left to rest overnight. It was then extracted with EtOAc (2×50 mL). The organics were washed with brine, dried over Na₂SO₄ and evaporated to give 4-methyl-5-(1-methylethyl)-2H-1,3-oxazine-2,6(3H)-dione (2.95 g, 17.44 mmol, 78% yield).

¹H-NMR (400 MHz, CHLOROFORM-d) d ppm 10.02 (br. s., 1H), 2.85 (spt, J=7.03 Hz, 1H), 2.20 (s, 3H), 1.25 (d, J=7.07 Hz, 6H)

4b) 3-[(4-Bromo-2-fluorophenyl)methyl]-4-methyl-5-(1-methylethyl)-2H-1,3-oxazine-2,6(3H)-dione. To a solution of 4-methyl-5-(1-methylethyl)-2H-1,3-oxazine-2,6(3H)-dione (750 mg, 4.43 mmol) in N,N-dimethylformamide (DMF) (7 ml) at 0° C. was added sodium hydride (266 mg, 6.65 mmol) in portions. The reaction mixture was stirred at room temperature for 5 minutes and then cooled to 0° C. and 4-bromo-1-(bromomethyl)-2-fluorobenzene (1188 mg, 4.43 mmol) was added portionwise. The mixture was stirred at rt for 18 h, then poured onto ice. The aqueous solution was extracted with ethyl acetate (2×100 ml). Organic layers were combined and washed with water (100 ml) and brine (100 ml), dried over sodium sulfate, filtered and the solvent evaporated to give a crude oil. It was purified by flash column chromatography (0-60% ethyl acetate in hexanes) to provide the title dione (3-[(4-bromo-2-fluorophenyl)methyl]-4-methyl-5-(1-methylethyl)-2H-1,3-oxazine-2,6(3H)-dione as an oil (190 mg, 0.507 mmol, 11.43% yield).

¹H NMR (400 MHz, CHLOROFORM-d) δ ppm 7.28-7.35 (m, 2H), 7.12 (t, J=8.21 Hz, 1 H), 5.09 (s, 2H), 2.90 (spt, J=7.03 Hz, 1H), 2.21 (s, 3H), 1.27 (d, 6H). LCMS (ES⁺) m/z 356 (MH⁺).

4c) N-{[1-[(4-bromo-2-fluorophenyl)methyl]-4-hydroxy-6-methyl-5-(1-methylethyl)-2-oxo-1,2-dihydro-3-pyridinyl]carbonyl}glycine. Sodium hydride (37.1 mg, 0.926 mmol) was suspended in DMF and the mixture was cooled at 0° C. Diethylmalonate (0.105 ml, 0.695 mmol) was added dropwise and stirring was continued for 10 minutes. A solution of 3-[(4-bromo-2-fluorophenyl)methyl]-4-methyl-5-(1-methylethyl)-2H-1,3-oxazine-2,6(3H)-dione (165 mg, 0.463 mmol) in 3.5 mL of DMF was then added. The reaction mixture was stirred at 120° C. for 3 hours, then cooled to 60° C. and treated with 1.5 mL AcOH. After stirrring for 10 minutes it was cooled to rt, poured onto H₂O and extracted with EtOAc. The organic layer was washed with 1N HCl, brine, dried over Na₂SO₄ and the solvent was evaporated. The residue was dissolved in ethanol (2.5 ml). Sodium glycinate (227 mg, 2.316 mmol) was added and the mixture was stirred at 145° C. for 20 min in a Biotage Initiator® microwave synthesizer. It was then poured onto H₂O and acidified by the addition of 1 N HCl (pH=2). The solid was collected, washed with H₂O and hexanes and purified on RP-HPLC (Gilson, 10 min gradient, 20 to 95% AcCN in H₂O plus 0.1% TFA). N-{[1-[(4-bromo-2-fluorophenyl)methyl]-4-hydroxy-6-methyl-5-(1-methylethyl)-2-oxo-1,2-dihydro-3-pyridinyl]carbonyl}glycine was obtained as a white powder (19.8 mg, 0.041 mmol, 8.92% yield).

1H NMR (400 MHz, DMSO-d₆) δ ppm 16.41 (s, 1H), 12.84 (br. s., 1H), 10.46 (t, J=5.56 Hz, 1H), 7.61 (dd, J=9.85, 1.77 Hz, 1H), 7.38 (dd, J=8.08, 1.52 Hz, 1H), 6.78 (t, J=8.21 Hz, 1 H), 5.31 (s, 2H), 4.04 (d, J=5.56 Hz, 2H), 3.12-3.31 (m, 1H), 2.34 (s, 3H), 1.27 (d, J=7.07 Hz, 6H). LCMS (ES⁺) m/z 456 (MH⁺).

### Example 5

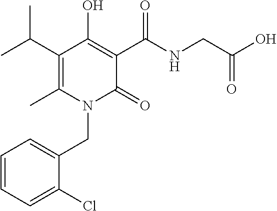

### N-{[1-[(2-chlorophenyl)methyl]-4-hydroxy-6-methyl-5-(1-methylethyl)-2-oxo-1,2-dihydro-3-pyridinyl]carbonyl}glycine

To a solution of 4-methyl-5-(1-methylethyl)-2H-1,3-oxazine-2,6(3H)-dione (770 mg, 4.55 mmol) in N,N-dimethylformamide (DMF) (7 ml) at 0° C. was added sodium hydride (273 mg, 6.83 mmol) in portions. The reaction mixture was stirred at room temperature for 5 minutes and then cooled down to 0° C. and 2-chlorobenzylbromide (0.886 ml, 6.83 mmol) was added. The mixture was stirred at rt for 18 h, then poured onto ice. The aqueous solution was extracted with ethyl acetate (2×100 ml), the organic layers combined and washed with brine, dried over Na₂SO₄, filtered and the solvent evaporated leaving a crude oil which was filtered through silica (0-70% EtOAc in hexanes). After evaporation of the solvent, the residue was dissolved in 3.5 mL of DMF and added to a cooled suspension of diethyl malonate (0.694 ml, 4.55 mmol) and sodium hydride (109 mg, 4.55 mmol) in 2 mL of DMF. The reaction mixture was stirred at 120° C. for 3 hours, then cooled to 60° C. and treated with 1.5 mL AcOH. After stirring for 10 minutes it was cooled to rt, poured onto H₂O and extracted with EtOAc. The organic layer was washed with 1N HCl, brine, dried over Na₂SO₄ and the solvent evaporated. The residue was purified on silica gel (2×: 0-60% EtOAc in hexanes, then 0-30% EtOAc in hexanes). The fractions containing product were evaporated and dissolved in ethanol (2.5 ml). Sodium glycinate (450 mg, 4.59 mmol) was added and the mixture was stirred at 140° C. for 25 min in a Biotage Initiator® microwave synthesizer. It was then poured onto H₂O and acidified by the addition of 1 N HCl (pH=2). The solid was collected, washed with H₂O and hexanes and purified on RP-HPLC (10 min. gradient, 25 to 95% AcCN in H₂O plus 0.1% TFA). The residue was taken up in water and collected by filtration to give N-{[1-[(2-chlorophenyl)methyl]-4-hydroxy-6-methyl-5-(1-methylethyl)-2-oxo-1,2-dihydro-3-pyridinyl]carbonyl}glycine as a white powder (43.5 mg, 0.109 mmol, 2.384% yield).

¹H-NMR (400 MHz, DMSO-d₆) δ ppm 16.42 (s, 1H), 12.84 (s, 1H), 10.47 (t, J=5.56 Hz, 1 H), 7.48-7.58 (m, 1H), 7.24-7.37 (m, 2H), 6.61-6.72 (m, 1H), 5.35 (s, 2H), 4.04 (d, J=5.56 Hz, 2H), 3.23 (sept, J=7.00 Hz, 1H), 2.31 (s, 3H), 1.29 (d, J=7.07 Hz, 6H). LCMS (ES⁺) m/z 393 (MH⁺).

### BIOLOGICAL BACKGROUND

The following references set out information about the target enzymes, HIF prolyl hydroxylases, and methods and materials for measuring inhibition of same by small molecules.
M. Hirsilä, P. Koivunen, V. Günzler, K. I. Kivirikko, and J. Myllyharju “Characterization of the Human Prolyl 4-Hydroxylases That Modify the Hypoxia-inducible Factor” J. Biol. Chem., 2003, 278, 30772-30780.
C. Willam, L. G. Nicholls, P. J. Ratcliffe, C. W. Pugh, P. H. Maxwell “The prolyl hydroxylase enzymes that act as oxygen sensors regulating destruction of hypoxia-inducible factor α” Advan. Enzyme Regul., 2004, 44, 75-92
M. S. Wiesener, J. S. Jiirgensen, C. Rosenberger, C. K. Scholze, J. H. Hörstrup, C. Warnecke, S. Mandriota, I. Bechmann, U. A. Frei, C. W. Pugh, P. J. Ratcliffe, S. Bachmann, P. H. Maxwell, and K.-U. Eckardt “Widespread hypoxia-inducible expression of HIF-2α in distinct cell populations of different organs” FASEB J., 2003, 17, 271-273.
S. J. Klaus, C. J. Molineaux, T. B. Neff, V. Guenzler-Pukall, I. Lansetmo Parobok, T. W. Seeley, R. C. Stephenson “Use of hypoxia-inducible factor α (HIFα) stabilizers for enhancing erythropoiesis” PCT Int. Appl. (2004), WO 2004108121 A1
C. Warnecke, Z. Zaborowska, J. Kurreck, V. A. Erdmann, U. Frei, M. Wiesener, and K.-U. Eckardt “Differentiating the functional role of hypoxia-inducible factor (HIF)-1α and HIF-2α (EPAS-1) by the use of RNA interference: erythropoietin is a HIF-2α target gene in Hep3B and Kelly cells” FASEB J., 2004, 18, 1462-1464.
   For the expression of EGLN3 see:
R. K. Bruick and S. L. McKnight “A Conserved Family of Prolyl-4-Hydroxylases That Modify HIF” Science, 2001, 294, 1337-1340.
   For the expression of HIF2α-CODD see:
a) P. Jaakkola, D. R. Mole, Y.-M. Tian, M. I. Wilson, J. Gielbert, S. J. Gaskell, A. von Kriegsheim, H. F. Hebestreit, M. Mukherji, C. J. Schofield, P. H. Maxwell, C. W. Pugh, P, J. Ratcliffe “Targeting of HIF-α to the von Hippel-Lindau Ubiquitylation Complex by O₂-Regulated Prolyl Hydroxylation” Science, 2001, 292, 468-472.
b) M. Ivan, K. Kondo, H. Yang, W. Kim, J. Valiando, M. Ohh, A. Salic, J. M. Asara, W. S. Lane, W. G. Kaelin Jr. “HIFα Targeted for VHL-Mediated Destruction by Proline Hydroxylation: Implications for O₂Sensing” Science, 2001, 292, 464-468.
   For the expression of VHL, elongin b and elongin c see:
A. Pause, S. Lee, R. A. Worrell, D. Y. T. Chen, W. H. Burgess, W. M. Linehan, R. D. Klausner “The von Hippel-Lindau tumor-suppressor gene product forms a stable complex with human CUL-2, a member of the Cdc53 family of proteins” Proc. Natl. Acad. Sci. USA, 1997, 94, 2156-2161.

### Biological Assay(s)

### EGLN3 Assay

### Materials:

His-MBP-EGLN3 (6HisMBPAttB1EGLN3(1-239)) was expressed in E. Coli and purified from an amylase affinity column. Biotin-VBC [6His SumoCysVHL(2-213), 6His SumoElonginB(1-118), and 6His SumoElonginC(1-112)] and His-GB1-HIF2α-CODD (6HisGB1tevHIF2A(467-572)) were expressed from E. Coli.

### Method:

Cy5-labelled HIF2α CODD, and a biotin-labeled VBC complex were used to determine EGLN3 inhibition. EGLN3 hydroxylation of the Cy5CODD substrate results in its recognition by the biotin-VBC. Addition of a Europium/streptavidin (Eu/SA) chelate results in proximity of Eu to Cy5 in the product, allowing for detection by energy transfer. A ratio of Cy5 to Eu emission (LANCE Ratio) is the ultimate readout, as this normalized parameter has significantly less variance than the Cy5 emission alone.

Then 50 nL of inhibitors in DMSO (or DMSO controls) were stamped into a 384-well low volume Corning NBS plate, followed by addition of 2.5 μL of enzyme [50 mL buffer (50 mM HEPES/50 mM KCl)+1 mL of a 10 mg/mL BSA in buffer+6.25 μL of a 10 mg/mL FeCl₂ solution in water+100 μL of a 200 mM solution of ascorbic acid in water+15.63 μL EGLN3] or control [50 mL buffer+1 mL of a 10 mg/mL BSA in buffer+6.25 μL of a 10 mg/mL FeCl₂ solution in water+100 μL of a 200 mM solution of ascorbic acid in water]. Following a 3 minutes incubation, 2.5 μL of substrate [50 mL Buffer+68.6 μL biotin-VBC+70.4 μL Eu (at 710 μg/mL stock)+91.6 μL Cy5CODD+50 μL of a 20 mM solution of 2-oxoglutaric acid in water+0.3 mM CHAPS] was added and incubated for 30 minutes. The plate was loaded into a PerkinElmer Viewlux for imaging. For dose response experiments, normalized data were fit by ABASE/XC50 using the equation y=a+(b−a)/(1+(10̂x/10̂c)Ad), where a is the minimum % activity, b is the maximum % activity, c is the pIC₅₀, and d is the Hill slope.

The IC₅₀ for exemplified compounds in the EGLN3 assay ranged from approximately 50-300 nanomolar. This range represents the data accumulated as of the time of the filing of this initial application. Later testing may show variations in IC₅₀ data due to variations in reagents, conditions and variations in the method(s) used from those given herein above. So this range is to be viewed as illustrative, and not a absolute set of numbers.

### Measure Epo Protein Produced by Hep3B Cell Line Using ELISA Method.

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37degC/5% CO₂/90% humidity (standard cell culture incubation conditions). After overnight adherence, medium is removed and replaced with DMEM without serum containing test compound or DMSO negative control. Following 48 hours incubation, cell culture medium is collected and assayed by ELISA to quantitate Epo protein.

The EC₅₀ for exemplar compounds in the Hep3B ELISA assay ranged from approximately 10-25 micromolar using the reagents and under the conditions outlined herein above. This range represents the data accumulated as of the time of the filing of this initial application. Later testing may show variations in EC₅₀ data due to variations in reagents, conditions and variations in the method(s) used from those given herein above. So this range is to be viewed as illustrative, and not a absolute set of numbers.

These compound are believed to be useful in therapy as defined above and to not have unacceptable or untoward effects when used in compliance with a permitted therapeutic regime.

The foregoing examples and assay have been set forth to illustrate the invention, not limit it. What is reserved to the inventors is to be determined by reference to the claims.

What is claimed is:

1. A compound of formula (I): 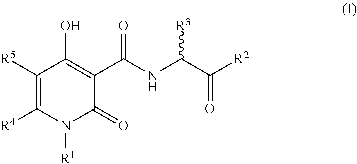
wherein:
R¹ is H, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;
R² is —NR⁶R⁷ or —OR⁸;
R³ is H or C₁-C₄alkyl;
R⁴ and R⁵ are each independently selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;
R⁶ and R⁷ are each independently selected from the group consisting of hydrogen, C₁-C₁₀ alkyl, C₂-C₁₀ alkenyl, C₂-C₁₀ alkynyl, C₃-C₈ cycloalkyl, C₃-C₈ heterocycloalkyl, aryl and heteroaryl;
R⁸ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R³, R⁴, R⁵, R⁶, R⁷, R⁸ is unsubstituted or, where possible, is substituted with one or more substituents independently selected from C₁-C₆ alkyl, aryl, heteroaryl, halogen, —OR⁹, —NR⁶R⁷, cyano, nitro, —C(O)R⁹, —C(O)OR⁹, —SR⁹, —S(O)R⁹, —S(O)₂R⁹, —NR⁶R⁷, —CONR⁶R⁷, —N(R⁶)C(O)R⁹, —N(R⁶)C(O)OR⁹, —OC(O)NR⁶R⁷, —N(R⁶)C(O)NR⁶R⁷, —SO₂NR⁶R⁷, —N(R⁶)SO₂R⁹, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R⁹ is hydrogen, C₁-C₁₀alkyl, C₂-C₁₀alkenyl, C₂-C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;
or a pharmaceutically acceptable salt or solvate thereof.

2. A compound according to claim 1 wherein:
R¹ is selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;
R² is —OR⁸;
R³ is H or C₁-C₄alkyl;
R⁴ and R⁵ are each independently selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;
R⁸ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R², R³, R⁴, R⁵, R⁸ is unsubstituted or,
where possible, is substituted with one or more substituents independently selected from C₁-C₆ alkyl, aryl, heteroaryl, halogen, —OR⁹, —NR⁶R⁷, cyano, nitro, —C(O)R⁹, —C(O)OR⁹, —SR⁹, —S(O)R⁹, —S(O)₂R⁹, —CONR⁶R⁷, —N(R⁶)C(O)R⁹, —N(R⁶)C(O)OR⁹, —OC(O)NR⁶R⁷, —N(R⁶)C(O)NR⁶R⁷, —SO₂NR⁶R⁷, —N(R⁶)SO₂R⁹, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R¹⁰ is hydrogen, C₁-C₁₀alkyl, C₂-C₁₀alkenyl, C₂-C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;
or a pharmaceutically acceptable salt or solvate thereof.

3. A compound according to claim 2 wherein:
R¹ and is selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;
R² is —OR⁸;
R³ is H;
R⁴ and R⁵ are each independently selected from the group consisting of hydrogen, —C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;
R⁸ is H or a cation;
where any carbon or heteroatom of R¹, R², R⁴, R⁵ is unsubstituted or, where possible, is substituted with one or more substituents independently selected from C₁-C₆ alkyl, aryl, heteroaryl, halogen, —OR⁹, —NR⁶R⁷, cyano, nitro, —C(O)R⁹, —C(O)OR⁹, —SR⁹, —S(O)R⁹, —S(O)₂R⁹, —CONR⁶R⁷, —N(R⁶)C(O)R⁹, —N(R⁶)C(O)OR⁹, —OC(O)NR⁶R⁷, —N(R⁶)C(O)NR⁶R⁷, —SO₂NR⁶R⁷, —N(R⁶)SO₂R⁹, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R⁹ is hydrogen, C₁-C₁₀alkyl, C₂-C₁₀alkenyl, C₂-C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;
or a pharmaceutically acceptable salt or solvate thereof.

4. A compound according to claim 1 which is:
N-{[4-hydroxy-5,6-dimethyl-2-oxo-1-(phenylmethyl)-1,2-dihydro-3-pyridinyl]carbonyl}glycine;
N-{[4-hydroxy-6-methyl-2-oxo-5-phenyl-1-(phenylmethyl)-1,2-dihydro-3-pyridinyl]carbonyl}glycine;
N-{[1-[(4-bromo-2-fluorophenyl)methyl]-4-hydroxy-6-methyl-5-(1-methylethyl)-2-oxo-1,2-dihydro-3-pyridinyl]carbonyl}glycine;
N-{[4-hydroxy-6-methyl-2-oxo-1,5-bis(phenylmethyl)-1,2-dihydro-3-pyridinyl]carbonyl}glycine;
N-{[1-[(2-chlorophenyl)methyl]-4-hydroxy-6-methyl-5-(1-methylethyl)-2-oxo-1,2-dihydro-3-pyridinyl]carbonyl}glycine;
or a pharmaceutically acceptable salt or solvate thereof.

5. A method for treating anemia in a mammal, which method comprises administering an effective amount of a compound of formula (I) or a salt or solvate thereof according to claim 1 to a mammalian suffering from anemia which can be treated by inhibiting HIF prolyl hydroxylases.

6. A pharmaceutical composition comprising a compound of formula (I) or a salt, solvate, according to claim 1 and one or more of pharmaceutically acceptable carriers, diluents and excipients.

7. A process for preparing a compound of formula (I) 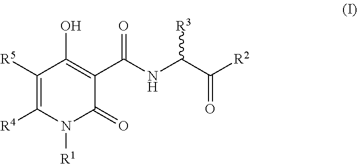
wherein:
R¹ is H, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;
R² is —NR⁶R⁷ or —OR⁸;
R³ is H or C₁-C₄alkyl;
R⁴ and R⁵ are each independently selected from the group consisting of hydrogen, C₁-C₁₀alkyl, C₂-C₁₀alkenyl, C₂-C₁₀alkynyl, C₃-C₈cycloalkyl, C₁-C₁₀alkyl-C₃-C₈cycloalkyl, C₅-C₈cycloalkenyl, C₁-C₁₀alkyl-C₅-C₈ cycloalkenyl, C₃-C₈ heterocycloalkyl, C₁-C₁₀alkyl-C₃-C₈ heterocycloalkyl, aryl, C₁-C₁₀alkyl-aryl, heteroaryl or C₁-C₁₀alkyl-heteroaryl;
R⁶ and R⁷ are each independently selected from the group consisting of hydrogen, C₁-C₁₀ alkyl, C₂-C₁₀ alkenyl, C₂-C₁₀ alkynyl, C₃-C₈ cycloalkyl, C₃-C₈ heterocycloalkyl, aryl and heteroaryl;
R⁸ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R³, R⁴, R⁵, R⁶, R⁷, R⁸ is unsubstituted or, where possible, is substituted with one or more substituents independently selected from C₁-C₆ alkyl, aryl, heteroaryl, halogen, —OR⁹, —NR⁶R⁷, cyano, nitro, —C(O)R⁹, —C(O)OR⁹, —SR⁹, —S(O)R⁹, —S(O)₂R⁹, —NR⁶R⁷, —CONR⁶R⁷, —N(R⁶)C(O)R⁹, —N(R⁶)C(O)OR⁹, —OC(O)NR⁶R⁷, —N(R⁶)C(O)NR⁶R⁷, —SO₂NR⁶R⁷, —N(R⁶)SO₂R⁹, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R⁹ is hydrogen, C₁-C₁₀alkyl, C₂-C₁₀alkenyl, C₂-C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;
or a pharmaceutically acceptable salt or solvate thereof.
comprising treating a compound of formula A: 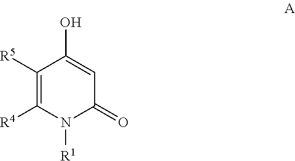
wherein R¹, R⁴ and R⁵ are the same as for those groups in formula (I) with an ethyl 2-isocyanatocarboxylate and an appropriate base, such as di-isopropylethylamine, in an appropriate solvent, such as dichloromethane, under either conventional thermal conditions or by microwave irradiation, to form a compound of formula (B) wherein R¹, R², R³, R⁴ and R⁵ are the same as for those groups in formula (I); 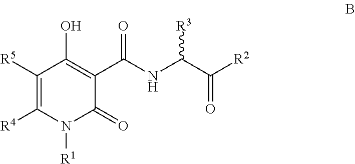
and treating the compound of formula (B) with an alkali such as sodium hydroxide, in an appropriate solvent, such as aqueous ethanol, at a suitable temperature such as room temperature, to form a compound of formula (I) where R² is —OH;
